(19) United States
(12) Patent Application Publication
(10) Pub. No.: US 2017/0355930 A1
(43) Pub. Date: Dec. 14, 2017

(54) CLEANING COMPOSITIONS INCLUDING NUCLEASE ENZYME AND AMINES

(71) Applicant: The Procter & Gamble Company, Cincinnati, OH (US)

(72) Inventor: Neil Joseph LANT, Newcastle upon Tyne (GB)

(21) Appl. No.: 15/613,359

(22) Filed: Jun. 5, 2017

Related U.S. Application Data
(60) Provisional application No. 62/347,653, filed on Jun. 9, 2016.

(51) Int. Cl. C11D 3/386; C11D 3/20; C11D 3/39; A61K 31/13; C11D 9/00; C11D 3/00

(52) U.S. Cl. CPC C11D 3/386; C11D 3/3932; A61K 31/13; C11D 3/2079; C11D 3/00; C11D 9/00

(57) ABSTRACT

Cleaning compositions that include a nuclease enzyme and an amine. Methods of making and using such cleaning compositions. Use of an amine. Use of a nuclease enzyme.

### FIELD OF THE INVENTION

The present disclosure relates to cleaning compositions that include a nuclease enzyme and an amine. The present disclosure also relates to methods of making and using such cleaning compositions. The present disclosure also relates to the use of an amine. The present disclosure also relates to the use of a nuclease enzyme.

### BACKGROUND OF THE INVENTION

The detergent formulator is constantly aiming to improve the performance of detergent compositions. One particular challenge is the removal of certain malodorous soils from surfaces such as textiles. Such soils may build up over time, including on collars and cuffs where incomplete cleaning may occur.

Nuclease enzymes, such as deoxyribonuclease enzymes, have been found to be effective on such soils, but their efficiency can be improved, particularly when such soils are present in combination with other soils, such as greasy soils.

There is a need for improved cleaning compositions that include nuclease enzymes.

### SUMMARY OF THE INVENTION

The present disclosure relates to cleaning compositions that include a nuclease enzyme and an amine.

The present disclosure relates to methods of cleaning a surface, such as a textile, that include mixing a cleaning composition as described herein with water to form an aqueous liquor and contacting a surface with the aqueous liquor in a laundering step.

The present disclosure also relates to the use of an amine to enhance the stain-removal and/or malodor-reducing benefits of a nuclease enzyme.

The present disclosure also relate to the use of a nuclease enzyme, preferably a deoxyribonuclease enzyme and/or a ribonuclease enzyme, to enhance the greasy-stain removal of an amine.

### DETAILED DESCRIPTION OF THE INVENTION

The present disclosure relates to cleaning compositions that include a nuclease enzyme and an amine, such as an etheramine. Without wishing to be bound by theory, it is believed that certain malodor-causing soils become trapped under greasy soils on certain surfaces, such as textiles. It is further believed that amines according to the present disclosure help to lift the greasy soils, facilitating the soil-removing (and malodor-reducing) benefits of the nucleases described herein.

The components of the compositions and processes of the present disclosure are described in more detail below.

As used herein, the articles “a” and “an” when used in a claim, are understood to mean one or more of what is claimed or described. As used herein, the terms “include,” “includes,” and “including” are meant to be non-limiting. The compositions of the present disclosure can comprise, consist essentially of, or consist of, the components of the present disclosure.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

As used herein, the term “etheramine” includes the term “polyetheramine” and includes amines that have one or more ether groups.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (° C.) unless otherwise indicated. Unless otherwise specified, all measurements herein are conducted at 20° C. and under the atmospheric pressure.

In all embodiments of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “alkoxy” is intended to include C1-C8 alkoxy and C1-C8 alkoxy derivatives of polyols having repeating units such as butylene oxide, glycidol oxide, ethylene oxide or propylene oxide.

As used herein, unless otherwise specified, the terms “alkyl” and “alkyl capped” are intended to include C1-C18 alkyl groups, or even C1-C6 alkyl groups.

As used herein, unless otherwise specified, the term “aryl” is intended to include C3-12 aryl groups.

As used herein, unless otherwise specified, the term “arylalkyl” and “alkaryl” are equivalent and are each intended to include groups comprising an alkyl moiety bound to an aromatic moiety, typically having C1-C18 alkyl groups and, in one aspect, C1-C6 alkyl groups.

The terms “ethylene oxide,” “propylene oxide” and “butylene oxide” may be shown herein by their typical designation of “EO,” “PO” and “BO,” respectively.

As used herein, the term “cleaning and/or treatment composition” includes, unless otherwise indicated, granular, powder, liquid, gel, paste, unit dose, bar form and/or flake type washing agents and/or fabric treatment compositions, including but not limited to products for laundering fabrics, fabric softening compositions, fabric enhancing compositions, fabric freshening compositions, and other products for the care and maintenance of fabrics, and combinations thereof. Such compositions may be pre-treatment compositions for use prior to a washing step or may be rinse added compositions, as well as cleaning auxiliaries, such as bleach additives and/or “stain-stick” or pre-treat compositions or substrate-laden products such as dryer added sheets.

As used herein, “cellulosic substrates” are intended to include any substrate which comprises cellulose, either 100% by weight cellulose or at least 20% by weight, or at least 30% by weight or at least 40 or at least 50% by weight or even at least 60% by weight cellulose. Cellulose may be found in wood, cotton, linen, jute, and hemp. Cellulosic substrates may be in the form of powders, fibers, pulp and articles formed from powders, fibers and pulp. Cellulosic fibers, include, without limitation, cotton, rayon (regenerated cellulose), acetate (cellulose acetate), triacetate (cellulose triacetate), and mixtures thereof. Typically cellulosic substrates comprise cotton. Articles formed from cellulosic fibers include textile articles such as fabrics. Articles formed from pulp include paper.

As used herein, the term “maximum extinction coefficient” is intended to describe the molar extinction coefficient at the wavelength of maximum absorption (also referred to herein as the maximum wavelength), in the range of 400 nanometers to 750 nanometers.

As used herein “average molecular weight” is reported as a weight average molecular weight, as determined by its molecular weight distribution; as a consequence of their manufacturing process, polymers disclosed herein may contain a distribution of repeating units in their polymeric moiety.

As used herein the term “variant” refers to a polypeptide that contains an amino acid sequence that differs from a wild type or reference sequence. A variant polypeptide can differ from the wild type or reference sequence due to a deletion, insertion, or substitution of a nucleotide(s) relative to said reference or wild type nucleotide sequence. The reference or wild type sequence can be a full-length native polypeptide sequence or any other fragment of a full-length polypeptide sequence. A polypeptide variant generally has at least about 70% amino acid sequence identity with the reference sequence, but may include 75% amino acid sequence identity within the reference sequence, 80% amino acid sequence identity within the reference sequence, 85% amino acid sequence identity with the reference sequence, 86% amino acid sequence identity with the reference sequence, 87% amino acid sequence identity with the reference sequence, 88% amino acid sequence identity with the reference sequence, 89% amino acid sequence identity with the reference sequence, 90% amino acid sequence identity with the reference sequence, 91% amino acid sequence identity with the reference sequence, 92% amino acid sequence identity with the reference sequence, 93% amino acid sequence identity with the reference sequence, 94% amino acid sequence identity with the reference sequence, 95% amino acid sequence identity with the reference sequence, 96% amino acid sequence identity with the reference sequence, 97% amino acid sequence identity with the reference sequence, 98% amino acid sequence identity with the reference sequence, 98.5% amino acid sequence identity with the reference sequence or 99% amino acid sequence identity with the reference sequence.

As used herein, the term “solid” includes granular, powder, bar and tablet product forms.

As used herein, the term “fluid” includes liquid, gel, paste, and gas product forms.

### Cleaning Composition

The present disclosure relates to cleaning compositions. The cleaning composition may be selected from the group of light duty liquid detergents compositions, heavy duty liquid detergent compositions, hard surface cleaning compositions, detergent gels commonly used for laundry, bleaching compositions, laundry additives, fabric enhancer compositions, shampoos, body washes, other personal care compositions, and mixtures thereof. The cleaning composition may be a hard surface cleaning composition (such as a dishwashing composition) or a laundry composition (such as a heavy duty liquid detergent composition).

The cleaning compositions may be in any suitable form. The composition can be selected from a liquid, solid, or combination thereof. As used herein, “liquid” includes free-flowing liquids, as well as pastes, gels, foams and mousses. Non-limiting examples of liquids include light duty and heavy duty liquid detergent compositions, fabric enhancers, detergent gels commonly used for laundry, bleach and laundry additives. Gases, e.g., suspended bubbles, or solids, e.g. particles, may be included within the liquids. A “solid” as used herein includes, but is not limited to, powders, agglomerates, and mixtures thereof. Non-limiting examples of solids include: granules, micro-capsules, beads, noodles, and pearlised balls. Solid compositions may provide a technical benefit including, but not limited to, through-the-wash benefits, pre-treatment benefits, and/or aesthetic effects.

The cleaning composition may be in the form of a unitized dose article, such as a tablet or in the form of a pouch. Such pouches typically include a water-soluble film, such as a polyvinyl alcohol water-soluble film, that at least partially encapsulates a composition. Suitable films are available from MonoSol, LLC (Indiana, USA). The composition can be encapsulated in a single or multi-compartment pouch. A multi-compartment pouch may have at least two, at least three, or at least four compartments. A multi-compartmented pouch may include compartments that are side-by-side and/or superposed. The composition contained in the pouch may be liquid, solid (such as powders), or combinations thereof.

### Nuclease Enzyme

The nuclease enzyme is an enzyme capable of cleaving the phosphodiester bonds between the nucleotide sub-units of nucleic acids. The nuclease enzyme herein is preferably a deoxyribonuclease or ribonuclease enzyme or a functional fragment thereof. By functional fragment or part is meant the portion of the nuclease enzyme that catalyzes the cleavage of phosphodiester linkages in the DNA backbone and so is a region of said nuclease protein that retains catalytic activity. Thus it includes truncated, but functional versions, of the enzyme and/or variants and/or derivatives and/or homologues whose functionality is maintained.

Preferably the nuclease enzyme is a deoxyribonuclease, preferably selected from any of the classes E.C. 3.1.21.x, where x=1, 2, 3, 4, 5, 6, 7, 8 or 9, E.C. 3.1.22.y where y=1, 2, 4 or 5, E.C. 3.1.30.z where z=1 or 2, E.C. 3.1.31.1 and mixtures thereof.

Nucleases in class E.C. 3.1.21.x cleave at the 3′ hydroxyl to liberate 5′ phosphomonoesters as follows:

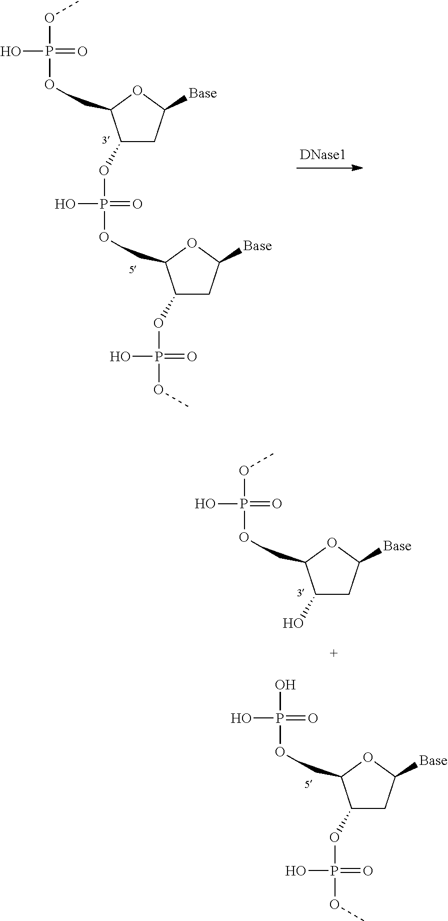

Nuclease enzymes from class E.C. 3.1.21.x and especially where x=1 are particularly preferred.

Nucleases in class E.C. 3.1.22.y cleave at the 5′ hydroxyl to liberate 3′ phosphomonoesters. Enzymes in class E.C. 3.1.30.z may be preferred as they act on both DNA and RNA and liberate 5′-phosphomonoesters. Suitable examples from class E.C. 3.1.31.2 are described in US2012/0135498A, such as SEQ ID NO:3 therein. Such enzymes are commercially available as DENARASE® enzyme from c-LECTA.

Nuclease enzymes from class E.C. 3.1.31.1 produce 3′ phosphomonoesters.

Preferably, the nuclease enzyme comprises a microbial enzyme. The nuclease enzyme may be fungal or bacterial in origin. Bacterial nucleases may be most preferred. Fungal nucleases may be most preferred.

The microbial nuclease is obtainable from Bacillus, such as a Bacillus licheniformis or Bacillus subtilis bacterial nucleases. A preferred nuclease is obtainable from Bacillus licheniformis, preferably from strain EI-34-6. A preferred deoxyribonuclease is a variant of Bacillus licheniformis, from strain EI-34-6 nucB deoxyribonuclease defined in SEQ ID NO: 1 herein, or variant thereof, for example having at least 70% or 75% or 80% or 85% or 90% or 95%, 96%, 97%, 98%, 99% or 100% identical thereto.

Other suitable nucleases are defined in SEQ ID NO:2 herein, or variant thereof, for example having at least 70% or 75% or 80% or 85% or 90% or 95%, 96%, 97%, 98%, 99% or 100% identical thereto. Other suitable nucleases are defined in SEQ ID NO:3 herein, or variant thereof, for example having at least 70% or 75% or 80% or 85% or 90% or 95%, 96%, 97%, 98%, 99% or 100% identical thereto.

A fungal nuclease is obtainable from Aspergillus, for example Aspergillus oryzae. A preferred nuclease is obtainable from Aspergillus oryzae defined in SEQ ID NO: 5 herein, or variant thereof, for example having at least 60% or 70% or 75% or 80% or 85% or 90% or 95%, 96%, 97%, 98%, 99% or 100% identical thereto.

Another suitable fungal nuclease is obtainable from Trichoderma, for example Trichoderma harzianum. A preferred nuclease is obtainable from Trichoderma harzianum defined in SEQ ID NO: 6 herein, or variant thereof, for example having at least 60% or 70% or 75% or 80% or 85% or 90% or 95%, 96%, 97%, 98%, 99% or 100% identical thereto.

Other fungal nucleases include those encoded by the DNA sequences of Aspergillus oryzae RIB40, Aspergillus oryzae 3.042, Aspergillus flavus NRRL3357, Aspergillus parasiticus SU-1, Aspergillus nomius NRRL13137, Trichoderma reesei QM6a, Trichoderma virens Gv29-8, Oidiodendron maius Zn, Metarhizium guizhouense ARSEF 977, Metarhizium majus ARSEF 297, Metarhizium robertsii ARSEF 23, Metarhizium acridum CQMa 102, Metarhizium brunneum ARSEF 3297, Metarhizium anisopliae, Colletotrichum fioriniae PJ7, Colletotrichum sublineola, Trichoderma atroviride IMI 206040, Tolypocladium ophioglossoides CBS 100239, Beauveria bassiana ARSEF 2860, Colletotrichum higginsianum, Hirsutella minnesotensis 3608, Scedosporium apiospermum, Phaeomoniella chlamydospora, Fusarium verticillioides 7600, Fusarium oxysporum f. sp. cubense race 4, Colletotrichum graminicola M1.001, Fusarium oxysporum FOSC 3-a, Fusarium avenaceum, Fusarium langsethiae, Grosmannia clavigera kw1407, Claviceps purpurea 20.1, Verticillium longisporum, Fusarium oxysporum f. sp. cubense race 1, Magnaporthe oryzae 70-15, Beauveria bassiana D1-5, Fusarium pseudograminearum CS3096, Neonectria ditissima, Magnaporthiopsis poae ATCC 64411, Cordyceps militaris CM01, Marssonina brunnea f. sp. ‘multigermtubi’ MB_m1, Diaporthe ampelina, Metarhizium album ARSEF 1941, Colletotrichum gloeosporioides Nara gc5, Madurella mycetomatis, Metarhizium brunneum ARSEF 3297, Verticillium alfalfae VaMs.102, Gaeumannomyces graminis var. tritici R3-111a-1, Nectria haematococca mpVI 77-13-4, Verticillium longisporum, Verticillium dahliae VdLs. 17, Torrubiella hemipterigena, Verticillium longisporum, Verticillium dahliae VdLs. 17, Botrytis cinerea B05.10, Chaetomium globosum CBS 148.51, Metarhizium anisopliae, Stemphylium lycopersici, Sclerotinia borealis F-4157, Metarhizium robertsii ARSEF 23, Myceliophthora thermophila ATCC 42464, Phaeosphaeria nodorum SN15, Phialophora attae, Ustilaginoidea virens, Diplodia seriata, Ophiostoma piceae UAMH 11346, Pseudogymnoascus pannorum VKM F-4515 (FW-2607), Bipolaris oryzae ATCC 44560, Metarhizium guizhouense ARSEF 977, Chaetomium thermophilum var. thermophilum DSM 1495, Pestalotiopsis fici W106-1, Bipolaris zeicola 26-R-13, Setosphaeria turcica Et28A, Arthroderma otae CBS 113480 and Pyrenophora tritici-repentis Pt-1C-BFP.

Preferably the nuclease is an isolated nuclease.

Preferably the nuclease enzyme is present in a the laundering aqueous solution in an amount of from 0.01 ppm to 1000 ppm of the nuclease enzyme, or from 0.05 or from 0.1 ppm to 750 or 500 ppm.

The nucleases may also give rise to biofilm-disrupting effects.

In a preferred composition, the composition additionally comprises a β-N-acetylglucosaminidase enzyme from E.C. 3.2.1.52, preferably an enzyme having at least 70%, or at least 75% or at least 80% or at least 85% or at least 90% or at least 95% or at least 96% or at least 97% or at least 98% or at least 99% or at least or 100% identity to SEQ ID NO:4.

### Amines

The cleaning compositions described herein may contain an amine. The cleaning compositions may include from about 0.1% to about 10%, or from about 0.2% to about 5%, or from about 0.5% to about 4%, or from about 0.1% to about 4%, or from about 0.1% to about 2%, by weight of the composition, of an amine. The amine can be subjected to protonation depending on the pH of the cleaning medium in which it is used.

Non-limiting examples of amines include, but are not limited to, etheramines, cyclic amines, polyamines, oligoamines (e.g., triamines, diamines, pentamines, tetraamines), or combinations thereof. The compositions described herein may comprise an amine selected from the group consisting of oligoamines, etheramines, cyclic amines, and combinations thereof. In some aspects, the amine is not an alkanolamine. In some aspects, the amine is not a polyalkyleneimine.

Examples of suitable oligoamines include tetraethylenepentamine, triethylenetetraamine, diethylenetriamine, and mixtures thereof. Etheramines and cyclic amines are described in more detail below.

### Etheramines

The cleaning compositions described herein may contain an etheramine. The cleaning compositions may contain from about 0.1% to about 10%, or from about 0.2% to about 5%, or from about 0.5% to about 4%, by weight of the composition, of an etheramine.

The etheramines of the present disclosure may have a weight average molecular weight of less than about grams/mole 1000 grams/mole, or from about 100 to about 800 grams/mole, or from about 200 to about 450 grams/mole, or from about 290 to about 1000 grams/mole, or from about 290 to about 900 grams/mole, or from about 300 to about 700 grams/mole, or from about 300 to about 450 grams/mole. The etheramines of the present invention may have a weight average molecular weight of from about 150, or from about 200, or from about 350, or from about 500 grams/mole, to about 1000, or to about 900, or to about 800 grams/mole.

The cleaning compositions may include an etheramine represented by the structure of Formula (I):

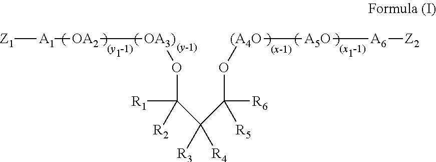

where each of R₁-R₆ is independently selected from H, alkyl, cycloalkyl, aryl, alkylaryl, or arylalkyl, where at least one of R₁-R₆ is different from H, typically at least one of R₁-R₆ is an alkyl group having 2 to 8 carbon atoms, each of A₁-A₆ is independently selected from linear or branched alkylenes having 2 to 18 carbon atoms, each of Z₁-Z₂ is independently selected from OH or NH₂, where at least one of Z₁-Z₂ is NH₂, typically each of Z₁ and Z₂ is NH₂, where the sum of x+y is in the range of about 2 to about 200, or about 2 to about 20, or about 2 to about 10, or about 2 to about 8, or about 3 to about 8, or about 4 to about 6, where x≧1 and y≧1, and the sum of x₁+y₁ is in the range of about 2 to about 200, or about 2 to about 20, or about 2 to about 10, or about 2 to about 8, or about 3 to about 8, or about 2 to about 4, where x₁≧1 and y₁≧1.

In the etheramine of Formula (I), each of A₁-A₆ may be independently selected from ethylene, propylene, or butylene, typically each of A₁-A₆ is propylene. Each of A₁ and A₆ may be independently selected from linear alkanediyl groups having 2 to 18 carbon atoms, or 2-10 carbon atoms, or 2-5 carbon atoms; each of A₂, A₃, A₄, and A₅ may be independently selected from linear or branched alkanediyl groups having 2 to 18 carbon atoms, preferably 2-10 carbon atoms, most preferably 2-5 carbon atoms. In the etheramine of Formula (I), each of R₁, R₂, R₅, and R₆ may be H and each of R₃ and R₄ may be independently selected from C1-C16 alkyl or aryl, typically each of R₁, R₂, R₅, and R₆ is H and each of R₃ and R₄ is independently selected from a butyl group, an ethyl group, a methyl group, a propyl group, or a phenyl group. In the etheramine of Formula (I), R₃ may be an ethyl group, each of R₁, R₂, R₅, and R₆ may be H, and R₄ may be a butyl group. In the etheramine of Formula (I), each of R₁ and R₂ may be H and each of R₃, R₄, R₅, and R₆ may be independently selected from an ethyl group, a methyl group, a propyl group, a butyl group, a phenyl group, or H.

The cleaning compositions described herein may include an etheramine represented by the structure of Formula (II):

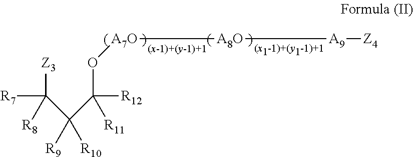

each of R₇-R₁₂ is independently selected from H, alkyl, cycloalkyl, aryl, alkylaryl, or arylalkyl, where at least one of R₇-R₁₂ is different from H, typically at least one of R₇-R₁₂ is an alkyl group having 2 to 8 carbon atoms, each of A₇-A₉ is independently selected from linear or branched alkylenes having 2 to 18 carbon atoms, each of Z₃-Z₄ is independently selected from OH or NH₂, where at least one of Z₃-Z₄ is NH₂, typically each of Z₃ and Z₄ is NH₂, where the sum of x+y is in the range of about 2 to about 200, or about 2 to about 20, or about 2 to about 10, or about 2 to about 8, or about 3 to about 8, or about 2 to about 4, where x≧1 and y≧1 and the sum of x₁+y₁ is in the range of about 2 to about 200, or about 2 to about 20, or about 2 to about 10, or about 2 to about 8, or about 3 to about 8, or about 2 to about 4, where x≧1 and y₁≧1.

In the etheramine of Formula (II), each of A₇-A₉ may be independently selected from ethylene, propylene, or butylene, typically each of A₇-A₉ is propylene. A₉ may be selected from linear alkanediyl groups having 2 to 18 carbon atoms, or 2-10 carbon atoms, or 2-5 carbon atoms; each of A₇ and A₈ may be independently selected from linear or branched alkanediyl groups having 2 to 18 carbon atoms, or 2-10 carbon atoms, or 2-5 carbon atoms. In the etheramine of Formula (II), each of R₇, R₈, R₁₁, and R₁₂ may be H and each of R₉ and R₁₀ may be independently selected from C1-C16 alkyl or aryl; each of R₇, R₈, R₁₁, and R₁₂ may be H and each of R₉ and R₁₀ may be independently selected from a butyl group, an ethyl group, a methyl group, a propyl group, or a phenyl group. In the etheramine of Formula (II), R₉ may be an ethyl group, each of R₇, R₈, R₁₁, and R₁₂ may be H, and R₁₀ may be a butyl group. In the etheramine of Formula (II), each of R₇ and R₈ may be H and each of R₉, R₁₀, R₁₁, and R₁₂ may be independently selected from an ethyl group, a methyl group, a propyl group, a butyl group, a phenyl group, or H.

Suitable etheramines are represented by Formula A, Formula B, and Formula C:

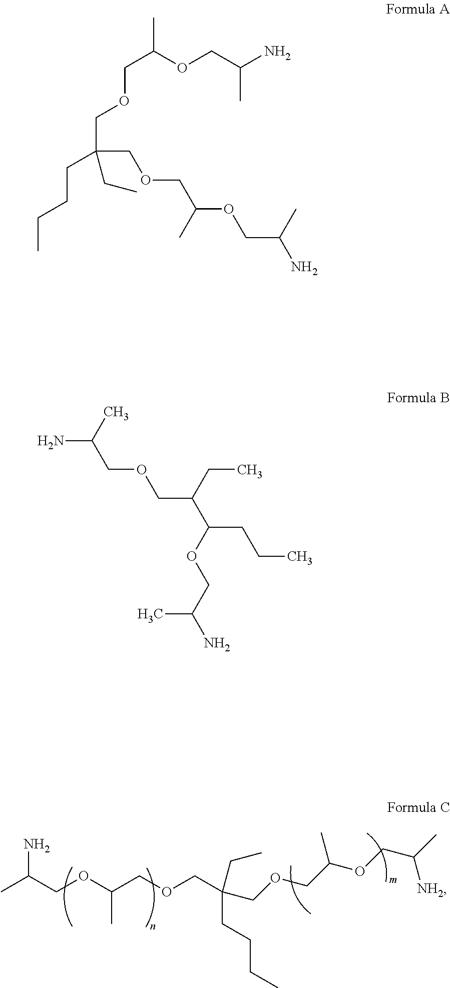

where n+m is from about 0 to about 8, or from about 0 to about 6, or from about 1 to about 6.

The etheramine comprises a mixture of the compound of Formula (I) and the compound of Formula (II).

The etheramine of Formula (I) or Formula (II) may have a weight average molecular weight of less than about grams/mole 1000 grams/mole, or from about 100 to about 900 grams/mole, or from about 100 to about 800 grams/mole, or from about 200 to about 450 grams/mole.

The etheramine can comprise a etheramine mixture comprising at least 90%, by weight of the etheramine mixture, of the etheramine of Formula (I), the etheramine of Formula(II), the etheramine of Formula(III) or a mixture thereof. The etheramine may comprise a etheramine mixture comprising at least 95%, by weight of the etheramine mixture, of the etheramine of Formula (I), the etheramine of Formula(II) and the etheramine of Formula(III).

The etheramine of Formula (I) and/or the etheramine of Formula (II) are obtainable by known methods, such as those disclosed in US2014/0296127A1. The etheramines of Formula (I) and/or Formula (II) may be obtained by:

a) reacting a 1,3-diol of formula (1) with a C₂-C₁₈ alkylene oxide to form an alkoxylated 1,3-diol, wherein the molar ratio of 1,3-diol to C₂-C₁₈ alkylene oxide is in the range of about 1:2 to about 1:10,

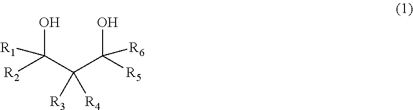

where R₁-R₆ are independently selected from H, alkyl, cycloalkyl, aryl, alkylaryl, or arylalkyl, where at least one of R₁-R₆ is different from H; and

b) aminating the alkoxylated 1,3-diol with ammonia.

Suitable 1,3-diols include 2,2-dimethyl-1,3-propane diol, 2-butyl-2-ethyl-1,3-propane diol, 2-pentyl-2-propyl-1,3-propane diol, 2-(2-methyl)butyl-2-propyl-1,3-propane diol, 2,2,4-trimethyl-1,3-propane diol, 2,2-diethyl-1,3-propane diol, 2-methyl-2-propyl-1,3-propane diol, 2-ethyl-1,3-hexane diol, 2-phenyl-2-methyl-1,3-propane diol, 2-methyl-1,3-propane diol, 2-ethyl-2-methyl-1,3 propane diol, 2,2-dibutyl-1,3-propane diol, 2,2-di(2-methylpropyl)-1,3-propane diol, 2-isopropyl-2-methyl-1,3-propane diol, or a mixture thereof. In some aspects, the 1,3-diol is selected from 2-butyl-2-ethyl-1,3-propanediol, 2-methyl-2-propyl-1,3-propanediol, 2-methyl-2-phenyl-1,3-propanediol, or a mixture thereof. Typically used 1,3-diols are 2-butyl-2-ethyl-1,3-propanediol, 2-methyl-2-propyl-1,3-propanediol, 2-methyl-2-phenyl-1,3-propanediol.

The degree of amination for the etheramine of Formula (I) and/or Formula (II) may be from about 50% to about 100%, or from about 60% to about 100%, or from about 70% to about 100%.

The degree of amination may be calculated from the total amine value (AZ) divided by sum of the total acetylables value (AC) and tertiary amine value (tert. AZ) multiplied by 100: (Total AZ/(AC+tert. AZ))×100). The total amine value (AZ) is determined according to DIN 16945. The total acetylables value (AC) is determined according to DIN 53240. The secondary and tertiary amines are determined according to ASTM D2074-07. The hydroxyl value is calculated from (total acetylables value+tertiary amine value)−total amine value.

The cleaning compositions of the present disclosure may contain an etheramine represented by Formula (III),

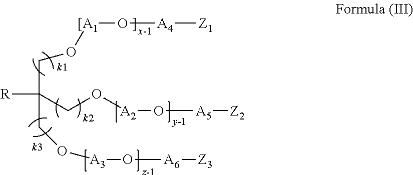

where
R is selected from H or a C1-C6 alkyl group,
each of k₁, k₂, and k₃ is independently selected from 0, 1, 2, 3, 4, 5, or 6,
each of A₁, A₂, A₃, A₄, A₅, and A₆ is independently selected from a linear or branched alkylene group having from about 2 to about 18 carbon atoms or mixtures thereof,
x≧1, y≧1, and z≧1, and the sum of x+y+z is in the range of from about 3 to about 100, and
each of Z₁, Z₂, and Z₃ is independently selected from NH₂ or OH, where at least two of Z₁, Z₂, and Z₃ are NH₂.

R may be H or a C1-C6 alkyl group selected from methyl, ethyl, or propyl. R may be H or a C1-C6 alkyl group selected from ethyl.

Each of k₁, k₂, and k₃ may be independently selected from 0, 1, or 2. Each of k₁, k₂, and k₃ may be independently selected from 0 or 1. At least two of k₁, k₂, and k₃ may be 1, or even each of k₁, k₂, and k₃ may be 1.

Each of Z₁, Z₂, and Z₃ may be NH₂.

All A groups (i.e., A₁-A₆) may be the same, at least two A groups may be the same, at least two A groups may be different, or all A groups may be different from each other. Each of A₁, A₂, A₃, A₄, A₅, and A₆ may be independently selected from a linear or branched alkylene group having from about 2 to about 10 carbon atoms, or from about 2 to about 6 carbon atoms, or from about 2 to about 4 carbon atoms, or mixtures thereof. At least one, or at least three, of A₁-A₆ may be a linear or branched butylene group. Each of A₄, A₅, and A₆ may be a linear or branched butylene group. Each of A₁-A₆ may be a linear or branched butylene group.

The variables x, y, and/or z may be independently selected and should be equal to 3 or greater, meaning that that the etheramine may have more than one [A₁-O] group, more than one [A₂-O] group, and/or more than one [A₃-O] group. A₁ may be selected from ethylene, propylene, butylene, or mixtures thereof. A₂ may be selected from ethylene, propylene, butylene, or mixtures thereof. A₃ may be selected from ethylene, propylene, butylene, or mixtures thereof. When A₁, A₂, and/or A₃ are mixtures of ethylene, propylene, and/or butylenes, the resulting alkoxylate may have a block-wise structure or a random structure.

[A₁-O]ₓ₋₁ can be selected from ethylene oxide, propylene oxide, butylene oxide, or mixtures thereof. [A₂-O]_{y-1} can be selected from ethylene oxide, propylene oxide, butylene oxide, or mixtures thereof. [A₃-O]_{z-1} can be selected from ethylene oxide, propylene oxide, butylene oxide, or mixtures thereof.

The sum of x+y+z may be in the range of from about 3 to about 100, or from about 3 to about 30, or from about 3 to about 10, or from about 5 to about 10.

When the etheramine is a etheramine of Formula (III) where R is a C2 alkyl group (i.e., ethyl) and optionally each of k₁, k₂, and k₃ is 1, the molecular weight of the etheramine may be from about 500 to about 1000, or to about 900, or to about 800 grams/mole. When the etheramine is an etheramine of Formula (III) where R is a C2 alkyl group (i.e., ethyl) and optionally each of k₁, k₂, and k₃ is 1, it may be that at least one A group (i.e., at least one of A1, A2, A3, A4, A5, or A6) is not a propylene group. When the etheramine is an etheramine of Formula (III) where R is a C2 alkyl group (i.e., ethyl) and optionally each of k₁, k₂, and k₃ is 1, it may be that at least one A group (i.e., at least one of A1, A2, A3, A4, A5, or A6) is an ethylene group or a butylene group, or even that at least one A group (i.e., at least one of A1, A2, A3, A4, A5, or A6) is a butylene group.

The compositions may include an etheramine selected from the group consisting of Formula D, Formula E, Formula F, and mixtures thereof:

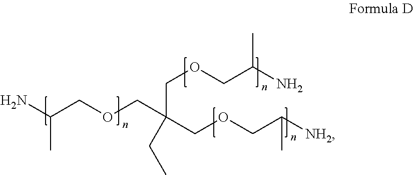

where average n is from about 0.5 to about 5, or about 1 to about 3, or about 1 to about 2.5;

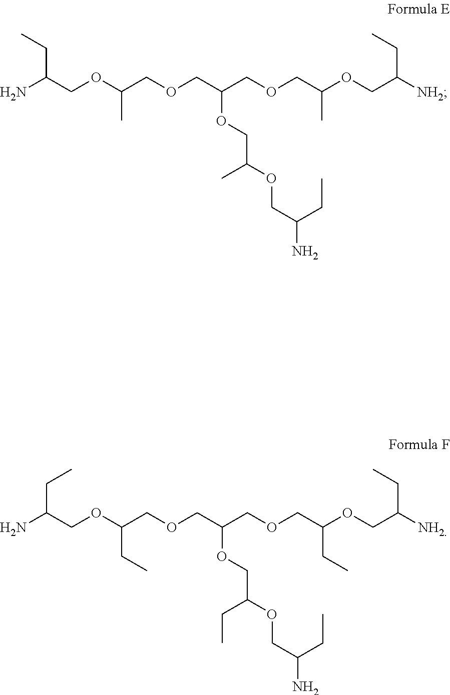 [SMILES: CCC(N)COC(C)COCC(COCC(C)OCC(N)CC)OCC(C)OCC(N)CC ; CCC(N)COC(CC)COCC(COCC(CC)OCC(N)CC)OCC(CC)OCC(N)CC]

The etheramines of Formula (III) are obtainable by known methods, such as those disclosed in US2015/0057212A1. The etheramines of Formula (III) may be obtained by a process comprising the following steps:

a) reacting a low-molecular-weight, organic triol, such as glycerine and/or 1,1,1-trimethylolpropane, with C₂-C₁₈ alkylene oxide, to form an alkoxylated triol, where the molar ratio of the low-molecular-weight organic triol to the alkylene oxide is in the range of about 1:3 to about 1:10, and

b) aminating the alkoxylated triol with ammonia.

The low-molecular-weight triol can be selected from glycerine, 1,1,1-trimethylolpropane, or mixtures thereof.

The etheramine of Formula (III) may have a weight average molecular weight of from about 500 to about 1000, or to about 900, or to about 800 grams/mole.

The degree of amination for the etheramine of Formula (III) may be may be from about 67% to about 100%, or from about 85% to about 100%. The degree of amination is calculated as described about in regard to the etheramines of Formula (I) and (II).

The cleaning compositions described herein may contain an etheramine as represented by the structure of Formula (IV):

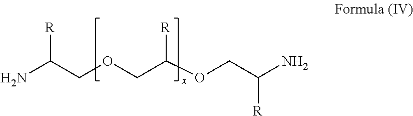

where each R group is independently selected from the group consisting of H, a methyl group, and an ethyl group, where at least one R group is a methyl group, x is in the range of about 2 to about 300. x indicates the average number of repeated units or basic building blocks that constitute the polymer. x may be a whole number or a fraction. x may be in the range of about 2 and about 20, or about 2 to about 10.

The primary amino groups of the etheramine of formula (IV) may be protonated, that is, ammonium groups. The etheramine according to the invention may comprise at least one repeated unit based on propylene oxide (R=a methyl group in formula (IV)) in the polymer backbone. The etheramine may have between about 2 and about 10 propylene oxide-based (PO) units. In the mentioned ranges (for the PO units), the hydrophobicity of the etheramine may provide for an improved cleaning on grease and particulate stains.

Suitable etheramines according to the invention are marketed by Huntsman Corp. Texas under the trade names, Jeffamine® D-230, Jeffamine® D-400, Jeffamine® ED-600, and by BASF under the trade names Baxxodur EC301, EC302.

The etheramine may be represented by the structure of Formula (E):

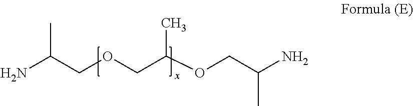

where x is about 2.5.

The etheramine of formula (IV) may have a weight average molecular weight of about 200 to about 1000 grams/mole, or about 230 to about 700 grams/mole, or about 230 to about 450 grams/mole.

The etheramine of Formula (IV) is obtainable by:

a) reacting a propane-1,2-diol of formula (2) with a C₂-C₁₈ alkylene oxide to form an alkoxylated propane-1,2-diol, wherein the molar ratio of propane-1,2-diol to C₂-C₁₈ alkylene oxide is in the range of about 1:2 to about 1:10,

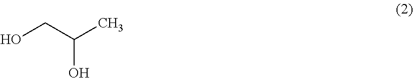

b) aminating the alkoxylated propane-1,2-diol with ammonia.

The degree of amination for the etheramine of Formula (IV) may be from about 50% to about 100%, typically from about 60% to about 100%, and more typically from about 70% to about 100%.

The degree of amination is calculated as described about in regard to the etheramines of Formula (I) and (II).

The etheramines of the invention are effective for removal of stains, particularly grease, from soiled material. Detergent compositions containing the etheramines of the invention also do not exhibit the cleaning negatives seen with conventional amine-containing detergent compositions on hydrophilic bleachable stains, such as coffee, tea, wine, or particulates. Additionally, unlike conventional amine-containing detergent compositions, the etheramines of the invention do not contribute to whiteness negatives on white fabrics. Furthermore, it is believed that the etheramines of the present disclosure are effective at facilitating nuclease enzyme efficacy.

The etheramines of the invention may be used in the form of a water-based, water-containing, or water-free solution, emulsion, gel or paste of the etheramine together with an acid such as, for example, citric acid, lactic acid, sulfuric acid, methanesulfonic acid, hydrogen chloride, e.g., aqueous hydrogen chloride, phosphoric acid, or mixtures thereof. Alternatively, the acid may be represented by a surfactant, such as, alkyl benzene sulfonic acid, alkylsulfonic acid, monoalkyl esters of sulphuric acid, mono alkylethoxy esters of sulphuric acid, fatty acids, alkyl ethoxy carboxylic acids, and the like, or mixtures thereof. When applicable or measurable, the preferred pH of the solution or emulsion ranges from pH 3 to pH 11, or from pH 6 to pH 9.5, even more preferred from pH 7 to pH 8.5.

Cyclic Amines

The cleaning compositions described herein may include a cyclic amine. The cleaning compositions may include from about 0.1% to about 10%, or from about 0.2% to about 5%, or from about 0.5% to about 3%, by weight the composition, of a cyclic amine.

The cyclic amine may be represented by the structure of Formula (V):

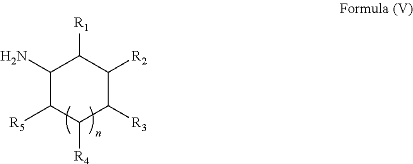

The substituents “R₅” may be independently selected from NH₂, H and linear, branched alkyl or alkenyl from 1 to 10 carbon atoms. For the purpose of this disclosure, “R₅” includes R1-R5. At least one of the “R₅” needs to be NH₂. The remaining “R₅” may be independently selected from NH₂, H and linear or branched alkyl or alkenyl having from 1 to 10 carbon atoms. n may be from 0 to 3; n may be 1.

The amine of the disclosure may be a cyclic amine with at least two primary amine functionalities. The primary amines can be in any position in the cycle but it has been found that in terms of grease cleaning, better performance may be obtained when the primary amines are in positions 1,3. It has also been found advantageous in terms of grease cleaning amines in which one of the substituents is —CH3 and the rest are H.

The term “cyclic amine” as used herein encompasses a single cyclic amine and a mixture thereof.

The cyclic amine can be subjected to protonation depending on the pH of the cleaning medium in which it is used.

### Adjuncts

The cleaning compositions described herein may include other adjunct components. The cleaning compositions may comprise a surfactant system as described below. The cleaning composition may comprise a fabric shading agent as described below and/or an additional enzyme selected from lipases, amylases, proteases, mannanases, pectate lyases, cellulases, cutinases, and mixtures thereof.

The composition may comprise a fabric shading agent. Suitable fabric shading agents include dyes, dye-clay conjugates, and pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof. Preferered dyes include alkoxylated azothiophenes, Solvent Violet 13, Acid Violet 50 and Direct Violet 9.

The cleaning compositions described herein may include one or more of the following non-limiting list of ingredients: fabric care benefit agent; detersive enzyme; deposition aid; rheology modifier; builder; chelant; bleach; bleaching agent; bleach precursor; bleach booster; bleach catalyst; perfume and/or perfume microcapsules; perfume loaded zeolite; starch encapsulated accord; polyglycerol esters; whitening agent; pearlescent agent; enzyme stabilizing systems; scavenging agents including fixing agents for anionic dyes, complexing agents for anionic surfactants, and mixtures thereof; optical brighteners or fluorescers; polymer including but not limited to soil release polymer and/or soil suspension polymer; dispersants; antifoam agents; non-aqueous solvent; fatty acid; suds suppressors, e.g., silicone suds suppressors; cationic starches; scum dispersants; substantive dyes; colorants; opacifier; antioxidant; hydrotropes such as toluenesulfonates, cumenesulfonates and naphthalenesulfonates; color speckles; colored beads, spheres or extrudates; clay softening agents; anti-bacterial agents. Additionally or alternatively, the compositions may comprise surfactants, quaternary ammonium compounds, and/or solvent systems. Quaternary ammonium compounds may be present in fabric enhancer compositions, such as fabric softeners, and comprise quaternary ammonium cations that are positively charged polyatomic ions of the structure NR₄⁺, where R is an alkyl group or an aryl group.

### Surfactant System

The cleaning composition may comprise a surfactant system. The cleaning composition may comprise from about 1% to about 80%, or from 1% to about 60%, preferably from about 5% to about 50% more preferably from about 8% to about 40%, by weight of the cleaning composition, of a surfactant system.

Surfactants of the present surfactant system may be derived from natural and/or renewable sources.

The surfactant system may comprise an anionic surfactant, more preferably an anionic surfactant selected from the group consisting of alkyl sulfate, alkyl alkoxy sulfate, especially alkyl ethoxy sulfate, alkyl benzene sulfonate, paraffin sulfonate and mixtures thereof. The surfactant system may further comprise a surfactant selected from the group consisting of nonionic surfactant, cationic surfactant, amphoteric surfactant, zwitterionic surfactant, and mixtures thereof. The surfactant system may comprise an amphoteric surfactant; the amphoteric surfactant may comprise an amine oxide surfactant. The surfactant system may comprise a nonionic surfactant; the nonionic surfactant may comprise an ethoxylated nonionic surfactant.

Alkyl sulfates are preferred for use herein and also alkyl ethoxy sulfates; more preferably a combination of alkyl sulfates and alkyl ethoxy sulfates with a combined average ethoxylation degree of less than 5, preferably less than 3, more preferably less than 2 and more than 0.5 and an average level of branching of from about 5% to about 40%.

The composition of the invention comprises amphoteric and/or zwitterionic surfactant, preferably the amphoteric surfactant comprises an amine oxide, preferably an alkyl dimethyl amine oxide, and the zwitteronic surfactant comprises a betaine surfactant.

The most preferred surfactant system for the detergent composition of the present invention comprise from 1% to 40%, preferably 6% to 35%, more preferably 8% to 30% weight of the total composition of an anionic surfactant, preferably an alkyl alkoxy sulfate surfactant, more preferably an alkyl ethoxy sulfate, combined with 0.5% to 15%, preferably from 1% to 12%, more preferably from 2% to 10% by weight of the composition of amphoteric and/or zwitterionic surfactant, more preferably an amphoteric and even more preferably an amine oxide surfactant, especially and alkyl dimethyl amine oxide. Preferably the composition further comprises a nonionic surfactant, especially an alcohol alkoxylate in particular and alcohol ethoxylate nonionic surfactant. It has been found that such surfactant system in combination with the polyetheramine of the invention provides excellent grease cleaning and good finish of the washed items.

Anionic Surfactant

Anionic surfactants include, but are not limited to, those surface-active compounds that contain an organic hydrophobic group containing generally 8 to 22 carbon atoms or generally 8 to 18 carbon atoms in their molecular structure and at least one water-solubilizing group preferably selected from sulfonate, sulfate, and carboxylate so as to form a water-soluble compound. Usually, the hydrophobic group will comprise a C8-C 22 alkyl, or acyl group. Such surfactants are employed in the form of water-soluble salts and the salt-forming cation usually is selected from sodium, potassium, ammonium, magnesium and mono-, di- or tri-C2-C3 alkanolammonium, with the sodium cation being the usual one chosen.

The anionic surfactant can be a single surfactant but usually it is a mixture of anionic surfactants. Preferably the anionic surfactant comprises a sulfate surfactant, more preferably a sulfate surfactant selected from the group consisting of alkyl sulfate, alkyl alkoxy sulfate and mixtures thereof. Preferred alkyl alkoxy sulfates for use herein are alkyl ethoxy sulfates.

Sulfated Anionic Surfactant

Preferably the sulfated anionic surfactant is alkoxylated, more preferably, an alkoxylated branched sulfated anionic surfactant having an alkoxylation degree of from about 0.2 to about 4, even more preferably from about 0.3 to about 3, even more preferably from about 0.4 to about 1.5 and especially from about 0.4 to about 1. Preferably, the alkoxy group is ethoxy. When the sulfated anionic surfactant is a mixture of sulfated anionic surfactants, the alkoxylation degree is the weight average alkoxylation degree of all the components of the mixture (weight average alkoxylation degree). In the weight average alkoxylation degree calculation the weight of sulfated anionic surfactant components not having alkoxylated groups should also be included.

Weight average alkoxylation degree=(x1*alkoxylation degree of surfactant 1+x2*alkoxylation degree of surfactant 2+ . . . )/(x1+x2+ . . . )

wherein x1, x2, . . . are the weights in grams of each sulfated anionic surfactant of the mixture and alkoxylation degree is the number of alkoxy groups in each sulfated anionic surfactant.

Preferably, the branching group is an alkyl. Typically, the alkyl is selected from methyl, ethyl, propyl, butyl, pentyl, cyclic alkyl groups and mixtures thereof. Single or multiple alkyl branches could be present on the main hydrocarbyl chain of the starting alcohol(s) used to produce the sulfated anionic surfactant used in the detergent of the invention. Most preferably the branched sulfated anionic surfactant is selected from alkyl sulfates, alkyl ethoxy sulfates, and mixtures thereof.

The branched sulfated anionic surfactant can be a single anionic surfactant or a mixture of anionic surfactants. In the case of a single surfactant the percentage of branching refers to the weight percentage of the hydrocarbyl chains that are branched in the original alcohol from which the surfactant is derived.

In the case of a surfactant mixture the percentage of branching is the weight average and it is defined according to the following formula:

Weight average of branching (%)=[(x1*wt % branched alcohol 1 in alcohol 1+x2*wt % branched alcohol 2 in alcohol 2+ . . . )/(x1+x2+ . . . )]*100

wherein x1, x2, . . . are the weight in grams of each alcohol in the total alcohol mixture of the alcohols which were used as starting material for the anionic surfactant for the detergent of the invention. In the weight average branching degree calculation the weight of anionic surfactant components not having branched groups should also be included.

Suitable sulfate surfactants for use herein include water-soluble salts of C8-C18 alkyl or hydroxyalkyl, sulfate and/or ether sulfate. Suitable counterions include alkali metal cation or ammonium or substituted ammonium, but preferably sodium.

The sulfate surfactants may be selected from C8-C18 primary, branched chain and random alkyl sulfates (AS); C8-C18 secondary (2,3) alkyl sulfates; C8-C18 alkyl alkoxy sulfates (AExS) wherein preferably x is from 1-30 in which the alkoxy group could be selected from ethoxy, propoxy, butoxy or even higher alkoxy groups and mixtures thereof.

Alkyl sulfates and alkyl alkoxy sulfates are commercially available with a variety of chain lengths, ethoxylation and branching degrees. Commercially available sulfates include, those based on Neodol alcohols ex the Shell company, Lial-Isalchem and Safol ex the Sasol company, natural alcohols ex The Procter & Gamble Chemicals company.

Preferably, the anionic surfactant comprises at least 50%, more preferably at least 60% and especially at least 70% of a sulfate surfactant by weight of the anionic surfactant. Especially preferred detergents from a cleaning view point are those in which the anionic surfactant comprises more than 50%, more preferably at least 60% and especially at least 70% by weight thereof of sulfate surfactant and the sulfate surfactant is selected from the group consisting of alkyl sulfates, alkyl ethoxy sulfates and mixtures thereof. Even more preferred are those in which the anionic surfactant is an alkyl ethoxy sulfate with a degree of ethoxylation of from about 0.2 to about 3, more preferably from about 0.3 to about 2, even more preferably from about 0.4 to about 1.5, and especially from about 0.4 to about 1. They are also preferred anionic surfactant having a level of branching of from about 5% to about 40%, even more preferably from about 10% to 35% and especially from about 20% to 30%.

Sulfonate Surfactant

Suitable anionic sulfonate surfactants for use herein include water-soluble salts of C8-C18 alkyl or hydroxyalkyl sulfonates; C11-C18 alkyl benzene sulfonates (LAS), modified alkylbenzene sulfonate (MLAS) as discussed in WO 99/05243, WO 99/05242, WO 99/05244, WO 99/05082, WO 99/05084, WO 99/05241, WO 99/07656, WO 00/23549, and WO 00/23548; methyl ester sulfonate (MES); and alpha-olefin sulfonate (AOS). Those also include the paraffin sulfonates may be monosulfonates and/or disulfonates, obtained by sulfonating paraffins of 10 to 20 carbon atoms. The sulfonate surfactant may also include the alkyl glyceryl sulfonate surfactants.

Nonionic Surfactant

Nonionic surfactant, when present, is comprised in a typical amount of from 0.1% to 40%, preferably 0.2% to 20%, most preferably 0.5% to 10% by weight of the composition. Suitable nonionic surfactants include the condensation products of aliphatic alcohols with from 1 to 25 moles of ethylene oxide. The alkyl chain of the aliphatic alcohol can either be straight or branched, primary or secondary, and generally contains from 8 to 22 carbon atoms. Particularly preferred are the condensation products of alcohols having an alkyl group containing from 10 to 18 carbon atoms, preferably from 10 to 15 carbon atoms with from 2 to 18 moles, preferably 2 to 15, more preferably 5-12 of ethylene oxide per mole of alcohol. Highly preferred nonionic surfactants are the condensation products of guerbet alcohols with from 2 to 18 moles, preferably 2 to 15, more preferably 5-12 of ethylene oxide per mole of alcohol.

Other suitable non-ionic surfactants for use herein include fatty alcohol polyglycol ethers, alkylpolyglucosides and fatty acid glucamides.

Amphoteric Surfactant

The surfactant system may include amphoteric surfactant, such as amine oxide. Preferred amine oxides are alkyl dimethyl amine oxide or alkyl amido propyl dimethyl amine oxide, more preferably alkyl dimethyl amine oxide and especially coco dimethyl amino oxide. Amine oxide may have a linear or mid-branched alkyl moiety. Typical linear amine oxides include water-soluble amine oxides containing one R1 C8-18 alkyl moiety and 2 R2 and R3 moieties selected from the group consisting of C1-3 alkyl groups and C1-3 hydroxyalkyl groups. Preferably amine oxide is characterized by the formula R1-N(R2)(R3) O wherein R1 is a C8-18 alkyl and R2 and R3 are selected from the group consisting of methyl, ethyl, propyl, isopropyl, 2-hydroxethyl, 2-hydroxypropyl and 3-hydroxypropyl. The linear amine oxide surfactants in particular may include linear C10-C18 alkyl dimethyl amine oxides and linear C8-C12 alkoxy ethyl dihydroxy ethyl amine oxides. Preferred amine oxides include linear C10, linear C10-C12, and linear C12-C14 alkyl dimethyl amine oxides. As used herein “mid-branched” means that the amine oxide has one alkyl moiety having n1 carbon atoms with one alkyl branch on the alkyl moiety having n2 carbon atoms. The alkyl branch is located on the a carbon from the nitrogen on the alkyl moiety. This type of branching for the amine oxide is also known in the art as an internal amine oxide. The total sum of n1 and n2 is from 10 to 24 carbon atoms, preferably from 12 to 20, and more preferably from 10 to 16. The number of carbon atoms for the one alkyl moiety (n1) should be approximately the same number of carbon atoms as the one alkyl branch (n2) such that the one alkyl moiety and the one alkyl branch are symmetric. As used herein “symmetric” means that |n1−n2| is less than or equal to 5, preferably 4, most preferably from 0 to 4 carbon atoms in at least 50 wt %, more preferably at least 75 wt % to 100 wt % of the mid-branched amine oxides for use herein. The amine oxide may further comprise two moieties, independently selected from a C1-3 alkyl, a C1-3 hydroxyalkyl group, or a polyethylene oxide group containing an average of from about 1 to about 3 ethylene oxide groups. Preferably the two moieties are selected from a C1-3 alkyl, more preferably both are selected as a C1 alkyl.

Zwitterionic Surfactant

Other suitable surfactants include betaines, such as alkyl betaines, alkylamidobetaine, amidazoliniumbetaine, sulfobetaine (INCI Sultaines) as well as the Phosphobetaine and preferably meets formula (I):

R—[CO—X(CH₂)ₙ]ₓ—N⁺(R²)(R₃)—(CH₂)ₘ—[CH(OH)—CH₂]_{y}—Y⁻ (I)

wherein
R¹ is a saturated or unsaturated C6-22 alkyl residue, preferably C8-18 alkyl residue, in particular a saturated C10-16 alkyl residue, for example a saturated C12-14 alkyl residue;
X is NH, NR⁴ with C1-4 Alkyl residue R⁴, O or S,
n a number from 1 to 10, preferably 2 to 5, in particular 3,
x 0 or 1, preferably 1,
R², R³ are independently a C1-4 alkyl residue, potentially hydroxy substituted such as a hydroxyethyl, preferably a methyl.
m a number from 1 to 4, in particular 1, 2 or 3,
y 0 or 1 and
Y is COO, SO3, OPO(OR⁵)O or P(O)(OR⁵)O, whereby R⁵ is a hydrogen atom H or a C1-4 alkyl residue.

Preferred betaines are the alkyl betaines of the formula (Ia), the alkyl amido propyl betaine of the formula (Ib), the Sulfo betaines of the formula (Ic) and the Amido sulfobetaine of the formula (Id);

R¹—N⁺(CH₃)₂—CH₂COO⁻ (Ia)

R¹—CO—NH(CH₂)₃—N⁺(CH₃)₂—CH₂COO⁻ (Ib)

R¹—N⁺(CH₃)₂—CH₂CH(OH)CH₂SO₃— (Ic)

R¹—CO—NH—(CH₂)₃—N⁺(CH₃)₂—CH₂CH(OH)CH₂SO₃— (Id) in which R¹1 as the same meaning as in formula I. Particularly preferred betaines are the Carbobetaine [wherein Y⁻═COO⁻], in particular the Carbobetaine of the formula (Ia) and (Ib), more preferred are the Alkylamidobetaine of the formula (Ib).

Examples of suitable betaines and sulfobetaine are the following [designated in accordance with INCI]: Almondamidopropyl of betaines, Apricotam idopropyl betaines, Avocadamidopropyl of betaines, Babassuamidopropyl of betaines, Behenam idopropyl betaines, Behenyl of betaines, betaines, Canolam idopropyl betaines, Capryl/Capram idopropyl betaines, Carnitine, Cetyl of betaines, Cocamidoethyl of betaines, Cocam idopropyl betaines, Cocam idopropyl Hydroxysultaine, Coco betaines, Coco Hydroxysultaine, Coco/Oleam idopropyl betaines, Coco Sultaine, Decyl of betaines, Dihydroxyethyl Oleyl Glycinate, Dihydroxyethyl Soy Glycinate, Dihydroxyethyl Stearyl Glycinate, Dihydroxyethyl Tallow Glycinate, Dimethicone Propyl of PG-betaines, Erucam idopropyl Hydroxysultaine, Hydrogenated Tallow of betaines, Isostearam idopropyl betaines, Lauram idopropyl betaines, Lauryl of betaines, Lauryl Hydroxysultaine, Lauryl Sultaine, Milkam idopropyl betaines, Minkamidopropyl of betaines, Myristam idopropyl betaines, Myristyl of betaines, Gleam idopropyl betaines, Gleam idopropyl Hydroxysultaine, Oleyl of betaines, Olivamidopropyl of betaines, Palmam idopropyl betaines, Palm itam idopropyl betaines, Palmitoyl Carnitine, Palm Kernelam idopropyl betaines, Polytetrafluoroethylene Acetoxypropyl of betaines, Ricinoleam idopropyl betaines, Sesam idopropyl betaines, Soyam idopropyl betaines, Stearam idopropyl betaines, Stearyl of betaines, Tallowam idopropyl betaines, Tallowam idopropyl Hydroxysultaine, Tallow of betaines, Tallow Dihydroxyethyl of betaines, Undecylenam idopropyl betaines and Wheat Germam idopropyl betaines.

A preferred betaine is, for example, Cocoamidopropylbetaine.

### Methods of Making the Composition

The present disclosure relates to methods of making the compositions described herein. The compositions of the invention may be solid (for example granules or tablets) or liquid form. Preferably the compositions are in liquid form. They may be made by any process chosen by the formulator, including by a batch process, a continuous loop process, or combinations thereof.

When in the form of a liquid, the compositions of the invention may be aqueous (typically above 2 wt % or even above 5 or 10 wt % total water, up to 90 or up to 80 wt % or 70 wt % total water) or non-aqueous (typically below 2 wt % total water content). Typically the compositions of the invention will be in the form of an aqueous solution or uniform dispersion or suspension of optical brightener, DTI and optional additional adjunct materials, some of which may normally be in solid form, that have been combined with the normally liquid components of the composition, such as the liquid alcohol ethoxylate nonionic, the aqueous liquid carrier, and any other normally liquid optional ingredients. Such a solution, dispersion or suspension will be acceptably phase stable. When in the form of a liquid, the detergents of the invention preferably have viscosity from 1 to 1500 centipoises (1-1500 mPa*s), more preferably from 100 to 1000 centipoises (100-1000 mPa*s), and most preferably from 200 to 500 centipoises (200-500 mPa*s) at 20 s−1 and 21° C. Viscosity can be determined by conventional methods. Viscosity may be measured using an AR 550 rheometer from TA instruments using a plate steel spindle at 40 mm diameter and a gap size of 500 am. The high shear viscosity at 20 s−1 and low shear viscosity at 0.05-1 can be obtained from a logarithmic shear rate sweep from 0.1-1 to 25-1 in 3 minutes time at 21 C. The preferred rheology described therein may be achieved using internal existing structuring with detergent ingredients or by employing an external rheology modifier. More preferably the detergents, such as detergent liquid compositions have a high shear rate viscosity of from about 100 centipoise to 1500 centipoise, more preferably from 100 to 1000 cps. Unit Dose detergents, such as detergent liquid compositions have high shear rate viscosity of from 400 to 1000 cps. Detergents such as laundry softening compositions typically have high shear rate viscosity of from 10 to 1000, more preferably from 10 to 800 cps, most preferably from 10 to 500 cps. Hand dishwashing compositions have high shear rate viscosity of from 300 to 4000 cps, more preferably 300 to 1000 cps.

The cleaning and/or treatment compositions in the form of a liquid herein can be prepared by combining the components thereof in any convenient order and by mixing, e.g., agitating, the resulting component combination to form a phase stable liquid detergent composition. In a process for preparing such compositions, a liquid matrix is formed containing at least a major proportion, or even substantially all, of the liquid components, e.g., nonionic surfactant, the non-surface active liquid carriers and other optional liquid components, with the liquid components being thoroughly admixed by imparting shear agitation to this liquid combination. For example, rapid stirring with a mechanical stirrer may usefully be employed. While shear agitation is maintained, substantially all of any anionic surfactants and the solid form ingredients can be added. Agitation of the mixture is continued, and if necessary, can be increased at this point to form a solution or a uniform dispersion of insoluble solid phase particulates within the liquid phase. After some or all of the solid-form materials have been added to this agitated mixture, particles of any enzyme material to be included, e.g., enzyme granulates, are incorporated. As a variation of the composition preparation procedure hereinbefore described, one or more of the solid components may be added to the agitated mixture as a solution or slurry of particles premixed with a minor portion of one or more of the liquid components. After addition of all of the composition components, agitation of the mixture is continued for a period of time sufficient to form compositions having the requisite viscosity and phase stability characteristics. Frequently this will involve agitation for a period of from about 30 to 60 minutes.

The adjunct ingredients in the compositions of this invention may be incorporated into the composition as the product of the synthesis generating such components, either with or without an intermediate purification step. Where there is no purification step, commonly the mixture used will comprise the desired component or mixtures thereof (and percentages given herein relate to the weight percent of the component itself unless otherwise specified) and in addition unreacted starting materials and impurities formed from side reactions and/or incomplete reaction. For example, for an ethoxylated or substituted component, the mixture will likely comprise different degrees of ethoxylation/substitution.

### Method of Use

The present disclosure relates to methods of using the cleaning compositions of the present disclosure to clean a surface, such as a textile. In general, the method includes mixing the cleaning composition as described herein with water to form an aqueous liquor and contacting a surface, preferably a textile, with the aqueous liquor in a laundering step. The target surface may include a greasy soil.

The compositions of this invention, typically prepared as hereinbefore described, can be used to form aqueous washing/treatment solutions for use in the laundering/treatment of fabrics and/or hard surfaces. Generally, an effective amount of such a composition is added to water, for example in a conventional fabric automatic washing machine, to form such aqueous laundering solutions. The aqueous washing solution so formed is then contacted, typically under agitation, with the fabrics to be laundered/treated therewith. An effective amount of the detergent composition herein added to water to form aqueous laundering solutions can comprise amounts sufficient to form from about 500 to 25,000 ppm, or from 500 to 15,000 ppm of composition in aqueous washing solution, or from about 1,000 to 3,000 ppm of the detergent compositions herein will be provided in aqueous washing solution.

Typically, the wash liquor is formed by contacting the detergent with wash water in such an amount so that the concentration of the detergent in the wash liquor is from above 0 g/l to 5 g/l, or from 1 g/l, and to 4.5 g/l, or to 4.0 g/l, or to 3.5 g/l, or to 3.0 g/l, or to 2.5 g/l, or even to 2.0 g/l, or even to 1.5 g/l. The method of laundering fabric or textile may be carried out in a top-loading or front-loading automatic washing machine, or can be used in a hand-wash laundry application. In these applications, the wash liquor formed and concentration of laundry detergent composition in the wash liquor is that of the main wash cycle. Any input of water during any optional rinsing step(s) is not included when determining the volume of the wash liquor.

The wash liquor may comprise 40 litres or less of water, or 30 litres or less, or 20 litres or less, or 10 litres or less, or 8 litres or less, or even 6 litres or less of water. The wash liquor may comprise from above 0 to 15 litres, or from 2 litres, and to 12 litres, or even to 8 litres of water. Typically from 0.01 kg to 2 kg of fabric per litre of wash liquor is dosed into said wash liquor. Typically from 0.01 kg, or from 0.05 kg, or from 0.07 kg, or from 0.10 kg, or from 0.15 kg, or from 0.20 kg, or from 0.25 kg fabric per litre of wash liquor is dosed into said wash liquor. Optionally, 50 g or less, or 45 g or less, or 40 g or less, or 35 g or less, or 30 g or less, or 25 g or less, or 20 g or less, or even 15 g or less, or even 10 g or less of the composition is contacted to water to form the wash liquor. Such compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric ratio is typically from about 1:1 to about 30:1. Typically the wash liquor comprising the detergent of the invention has a pH of from 3 to 11.5.

In one aspect, such method comprises the steps of optionally washing and/or rinsing said surface or fabric, contacting said surface or fabric with any composition disclosed in this specification then optionally washing and/or rinsing said surface or fabric is disclosed, with an optional drying step.

Drying of such surfaces or fabrics may be accomplished by any one of the common means employed either in domestic or industrial settings: machine drying or open-air drying. The fabric may comprise any fabric capable of being laundered in normal consumer or institutional use conditions, and the invention is particularly suitable for synthetic textiles such as polyester and nylon and especially for treatment of mixed fabrics and/or fibres comprising synthetic and cellulosic fabrics and/or fibres. As examples of synthetic fabrics are polyester, nylon, these may be present in mixtures with cellulosic fibres, for example, polycotton fabrics. The solution typically has a pH of from 7 to 11, more usually 8 to 10.5. The compositions are typically employed at concentrations from 500 ppm to 5,000 ppm in solution. The water temperatures typically range from about 5° C. to about 90° C. The water to fabric ratio is typically from about 1:1 to about 30:1.

### Use of an Amine

The present disclosure further relates to a use of an amine, such as an etheramine (e.g., a polyetheramine) to enhance the malodor-reducing benefits of a nuclease enzyme, such as a deoxyribonuclease enzyme and/or a ribonuclease enzyme.

### Use of a Nuclease Enzyme

The present disclosure further relates to a use of a nuclease enzyme, such as a deoxyribonuclease enzyme and/or a ribonuclease enzyme, to enhance the greasy-stain removal of an amine, such as an etheramine (e.g., a polyetheramine) as described above.

### COMBINATIONS

Specifically contemplated combinations of the disclosure are herein described in the following numbered paragraphs. These combinations are intended to be illustrative in nature and are not intended to be limiting.

### Examples

The following are illustrative examples of cleaning compositions according to the present disclosure and are not intended to be limiting.

### Examples 1 to 18: Unit Dose Compositions

These examples provide various formulations for unit dose laundry detergents and comprise double compartment unit dose products comprising one powder and one liquid compartment. The film used to encapsulate the compositions in PVA. Each example is prepared by combining a liquid compartment composition selected from compositions A-E with a powder compartment composition selected from compositions F-K.

| | Example | | | | | |
|---|---|---|---|---|---|---|
| | 1 | 2 | 3 | 4 | 5 | 6 |
| Liquid | 20 g A | 25 g A | 20 g A | 15 g A | 20 g B | 20 g B |
| composition | | | | | | |
| Solid | 15 g F | 12 g G | 12 g H | 12 g I | 15 g J | 15 g K |
| composition | | | | | | |

| | Example | | | | | |
|---|---|---|---|---|---|---|
| | 7 | 8 | 9 | 10 | 11 | 12 |
| Liquid | 15 g B | 17 g B | 20 g C | 19 g C | 15 g C | 25 g C |
| composition | | | | | | |
| Solid | 15 g L | 14 g F | 15 g G | 18 g H | 15 g I | 12 g J |
| composition | | | | | | |

| | Example | | | | | |
|---|---|---|---|---|---|---|
| | 13 | 14 | 15 | 16 | 17 | 18 |
| Liquid | 20 g D | 18 g D | 22 g D | 32 g E | 32 g E | 27 g E |
| composition | | | | | | |
| Solid | 20 g K | 13 g L | 15 g F | 17 g G | 12 g H | 18 g I |
| composition | | | | | | |

| | A | B | C | D | E |
|---|---|---|---|---|---|
| Ingredients | % weight of compartment | | | | |
| LAS | 19.09 | 16.76 | 8.59 | 6.56 | 3.44 |
| AE3S | 1.91 | 0.74 | 0.18 | 0.46 | 0.07 |
| AE7 | 14.00 | 17.50 | 26.33 | 28.08 | 31.59 |
| Citric Acid | 0.6 | 0.6 | 0.6 | 0.6 | 0.6 |
| C12-15 Fatty Acid | 14.8 | 14.8 | 14.8 | 14.8 | 14.8 |
| Polymer 3 | 4.0 | 4.0 | 4.0 | 4.0 | 4.0 |
| Chelant 2 | 1.2 | 1.2 | 1.2 | 1.2 | 1.2 |
| Optical Brightener 1 | 0.20 | 0.25 | 0.01 | 0.01 | 0.50 |
| Optical Brightener 2 | 0.20 | — | 0.25 | 0.03 | 0.01 |
| Optical Brightener 3 | 0.18 | 0.09 | 0.30 | 0.01 | — |
| DTI 1 | 0.10 | — | 0.20 | 0.01 | 0.05 |
| DTI 2 | — | 0.10 | 0.20 | 0.25 | 0.05 |
| Glycerol | 6.1 | 6.1 | 6.1 | 6.1 | 6.1 |
| Monoethanol amine | 8.0 | 8.0 | 8.0 | 8.0 | 8.0 |
| Tri-isopropanol amine | — | — | 2.0 | — | — |
| Tri-ethanol amine | — | 2.0 | — | — | — |
| Cumene sulfonate | — | — | — | — | 2.0 |
| Protease | 0.80 | 0.60 | 0.07 | 1.00 | 1.50 |
| Mannanase | 0.07 | 0.05 | 0.05 | 0.10 | 0.01 |
| Amylase 1 | 0.20 | 0.11 | 0.30 | 0.50 | 0.05 |
| Amylase 2 | 0.11 | 0.20 | 0.10 | — | 0.50 |
| Polishing enzyme | 0.005 | 0.05 | — | — | — |
| Nuclease | 0.005 | 0.05 | 0.005 | 0.010 | 0.005 |
| Dispersin B | 0.010 | 0.05 | 0.005 | 0.005 | — |
| Cyclohexyl dimethanol | — | — | — | 2.0 | — |
| Acid violet 50 | 0.03 | 0.02 | | | |
| Violet DD | | | 0.01 | 0.05 | 0.02 |
| Structurant | 0.14 | 0.14 | 0.14 | 0.14 | 0.14 |
| Perfume | 1.9 | 1.9 | 1.9 | 1.9 | 1.9 |

| | |
|---|---|
| Water, solvents and | To 100% |
| miscellaneous | |
| pH | 7.5-8.2 |

| | F | G | H | I | J | K |
|---|---|---|---|---|---|---|
| Ingredient | % weight | | | | | |
| Sodium carbonate | 20.0 | 35.0 | 30.0 | 29.0 | 28.0 | 18.0 |
| Carboxymethyl cellulose | 2.0 | 1.0 | — | — | 2.5 | 0.6 |
| Sodium silicate 2R | 5.0 | — | 5.0 | 3.2 | 20.0 | — |
| Tetraacetyl ethylenediamine | 20.0 | 15.0 | 18.0 | 15.0 | — | 25.0 |
| Sodium percarbonate | 50.0 | 44.0 | 45.0 | 45.0 | 29.0 | 50.0 |
| Polyetheramine | 0.5 | 2 | 0.5 | 1 | 0.5 | 4 |

| | |
|---|---|
| Sulfate/Water & | Balance |
| Miscellaneous | |

Based on total cleaning and/or treatment composition/compartment weight. Enzyme levels are reported as raw material.

### Examples 19 to 24: Granular Laundry Detergent Compositions for Hand Washing or Washing Machines, Typically Top-Loading Washing Machines

| | 19 | 20 | 21 | 22 | 23 | 24 |
|---|---|---|---|---|---|---|
| Ingredient | % weight | | | | | |
| LAS | 11.33 | 10.81 | 7.04 | 4.20 | 3.92 | 2.29 |
| Quaternary ammonium | 0.70 | 0.20 | 1.00 | 0.60 | — | — |
| AE3S | 0.51 | 0.49 | 0.32 | — | 0.08 | 0.10 |
| AE7 | 8.36 | 11.50 | 12.54 | 11.20 | 16.00 | 21.51 |
| Sodium Tripolyphosphate | 5.0 | — | 4.0 | 9.0 | 2.0 | — |
| Zeolite A | — | 1.0 | — | 1.0 | 4.0 | 1.0 |
| Sodium silicate 1.6R | 7.0 | 5.0 | 2.0 | 3.0 | 3.0 | 5.0 |
| Sodium carbonate | 20.0 | 17.0 | 23.0 | 14.0 | 14.0 | 16.0 |
| Polyacrylate MW 4500 | 1.0 | 0.6 | 1.0 | 1.0 | 1.5 | 1.0 |
| Polymer 6 | 0.1 | 0.2 | — | — | 0.1 | — |
| Carboxymethyl cellulose | 1.0 | 0.3 | 1.0 | 1.0 | 1.0 | 1.0 |
| Acid Violet 50 | 0.05 | — | 0.02 | — | 0.04 | — |
| Violet DD | — | 0.03 | — | 0.03 | — | 0.03 |
| Protease 2 | 0.10 | 0.10 | 0.10 | 0.10 | — | 0.10 |
| Amylase | 0.03 | — | 0.03 | 0.03 | 0.03 | 0.03 |
| Lipase | 0.03 | 0.07 | 0.30 | 0.10 | 0.07 | 0.40 |
| Polishing enzyme | 0.002 | — | 0.05 | — | 0.02 | — |
| Nuclease | 0.001 | 0.001 | 0.01 | 0.05 | 0.002 | 0.02 |
| Dispersin B | 0.001 | 0.001 | 0.05 | — | 0.001 | — |
| Optical Brightener 1 | 0.200 | 0.001 | 0.300 | 0.650 | 0.050 | 0.001 |
| Optical Brightener 2 | 0.060 | — | 0.650 | 0.180 | 0.200 | 0.060 |
| Optical Brightener 3 | 0.100 | 0.060 | 0.050 | — | 0.030 | 0.300 |
| Chelant 1 | 0.60 | 0.80 | 0.60 | 0.25 | 0.60 | 0.60 |
| DTI 1 | 0.32 | 0.15 | 0.15 | — | 0.10 | 0.10 |
| DTI 2 | 0.32 | 0.15 | 0.30 | 0.30 | 0.10 | 0.20 |
| Sodium Percarbonate | 4.6 | 5.2 | 5.0 | 5.7 | 4.5 | 7.3 |
| Nonanoyloxybenzensulfonate | 1.9 | 0.0 | 1.66 | 0.0 | 0.33 | 0.75 |
| Tetraacetylethylenediamine | 0.58 | 1.2 | 0.51 | 0.0 | 0.015 | 0.28 |
| Photobleach | 0.0030 | 0.0 | 0.0012 | 0.0030 | 0.0021 | — |
| S-ACMC | 0.1 | 0.0 | 0.0 | 0.0 | 0.06 | 0.0 |
| Polyetheramine | 0.5 | 2 | 0.5 | 1 | 0.5 | 4 |

| | |
|---|---|
| Sulfate/Moisture | Balance |

### Examples 25-30: Granular Laundry Detergent Compositions Typically for Front-Loading Automatic Washing Machines

| | 25 | 26 | 27 | 28 | 29 | 30 |
|---|---|---|---|---|---|---|
| Ingredient | % weight | | | | | |
| LAS | 6.08 | 5.05 | 4.27 | 3.24 | 2.30 | 1.09 |
| AE3S | — | 0.90 | 0.21 | 0.18 | — | 0.06 |
| AS | 0.34 | — | — | — | — | — |
| AE7 | 4.28 | 5.95 | 6.72 | 7.98 | 9.20 | 10.35 |
| Quaternary ammonium | 0.5 | — | — | 0.3 | — | — |
| Crystalline layered silicate | 4.1 | — | 4.8 | — | — | — |
| Zeolite A | 5.0 | — | 2.0 | — | 2.0 | 2.0 |
| Citric acid | 3.0 | 4.0 | 3.0 | 4.0 | 2.5 | 3.0 |
| Sodium carbonate | 11.0 | 17.0 | 12.0 | 15.0 | 18.0 | 18.0 |
| Sodium silicate 2R | 0.08 | — | 0.11 | — | — | — |
| Optical Brightener 1 | — | 0.25 | 0.05 | 0.01 | 0.10 | 0.02 |
| Optical Brightener 2 | — | — | 0.25 | 0.20 | 0.01 | 0.08 |
| Optical Brightener 3 | — | 0.06 | 0.04 | 0.15 | — | 0.05 |
| DTI 1 | 0.08 | — | 0.04 | — | 0.10 | 0.01 |
| DTI 2 | 0.08 | — | 0.04 | 0.10 | 0.10 | 0.02 |
| Soil release agent | 0.75 | 0.72 | 0.71 | 0.72 | — | — |
| Acrylic/maleic acid copolymer | 1.1 | 3.7 | 1.0 | 3.7 | 2.6 | 3.8 |
| Carboxymethyl cellulose | 0.2 | 1.4 | 0.2 | 1.4 | 1.0 | 0.5 |
| Protease 3 | 0.20 | 0.20 | 0.30 | 0.15 | 0.12 | 0.13 |
| Amylase 3 | 0.20 | 0.15 | 0.20 | 0.30 | 0.15 | 0.15 |
| Lipase | 0.05 | 0.15 | 0.10 | — | — | — |
| Amylase 2 | 0.03 | 0.07 | — | — | 0.05 | 0.05 |
| Cellulase 2 | — | — | — | — | 0.10 | 0.10 |
| Polishing enzyme | 0.003 | 0.005 | 0.020 | — | — | — |
| Nuclease | 0.002 | 0.010 | 0.020 | 0.020 | 0.010 | 0.003 |
| Dispersin B | 0.002 | 0.010 | 0.020 | 0.020 | 0.010 | 0.002 |
| Tetraacetylehtylenediamine | 3.6 | 4.0 | 3.6 | 4.0 | 2.2 | 1.4 |
| Sodium percabonate | 13.0 | 13.2 | 13.0 | 13.2 | 16.0 | 14.0 |
| Chelant 3 | — | 0.2 | — | 0.2 | — | 0.2 |
| Chelant 2 | 0.2 | — | 0.2 | — | 0.2 | 0.2 |
| MgSO₄ | — | 0.42 | — | 0.42 | — | 0.4 |
| Perfume | 0.5 | 0.6 | 0.5 | 0.6 | 0.6 | 0.6 |
| Suds suppressor agglomerate | 0.05 | 0.10 | 0.05 | 0.10 | 0.06 | 0.05 |
| Soap | 0.45 | 0.45 | 0.45 | 0.45 | — | — |
| Acid Violet 50 | 0.04 | — | 0.05 | — | 0.04 | — |
| Violet DD | — | 0.04 | — | 0.05 | — | 0.04 |
| S-ACMC | 0.01 | 0.01 | — | 0.01 | — | — |
| Direct Violet 9 (active) | — | — | 0.0001 | 0.0001 | — | — |
| Polyetheramine | 0.5 | 2 | 0.5 | 1 | 0.5 | 4 |

| | |
|---|---|
| Sulfate/Water & Miscellaneous | Balance |

AE1.8S is C₁₂₋₁₅ alkyl ethoxy (1.8) sulfate
AE3S is C₁₂₋₁₅ alkyl ethoxy (3) sulfate
AE7 is C₁₂₋₁₃ alcohol ethoxylate, with an average degree of ethoxylation of 7
AE8 is C₁₂₋₁₃ alcohol ethoxylate, with an average degree of ethoxylation of 8
AE9 is C₁₂₋₁₃ alcohol ethoxylate, with an average degree of ethoxylation of 9
Amylase 1 is Stainzyme®, 15 mg active/g
Amylase 2 is Natalase®, 29 mg active/g
Amylase 3 is Stainzyme® Plus, 20 mg active/g,
AS is C₁₂₋₁₄ alkylsulfate
Cellulase 2 is Celluclean™, 15.6 mg active/g
Xyloglucanase is Whitezyme®, 20 mg active/g
Chelant 1 is diethylene triamine pentaacetic acid
Chelant 2 is 1-hydroxyethane 1,1-diphosphonic acid
Chelant 3 is sodium salt of ethylenediamine-N,N′-disuccinic acid, (S,S) isomer (EDDS)
Dispersin B is a glycoside hydrolase, reported as 1000 mg active/g
DTI 1 is poly(4-vinylpyridine-1-oxide) (such as Chromabond S-403E®),
DTI 2 is poly(1-vinylpyrrolidone-co-1-vinylimidazole) (such as Sokalan HP56®).
HSAS is mid-branched alkyl sulfate as disclosed in U.S. Pat. No. 6,020,303 and U.S. Pat. No. 6,060,443
LAS is linear alkylbenzenesulfonate having an average aliphatic carbon chain
length C₉-C₁₅ (HLAS is acid form).
Lipase is Lipex®, 18 mg active/g
Mannanase is Mannaway®, 25 mg active/g
Nuclease is a Phosphodiesterase SEQ ID NO 1, reported as 1000 mg active/g
Optical Brightener 1 is disodium 4,4′-bis {[4-anilino-6-morpholino-s-triazin-2-yl]-amino}-2,2′-stilbenedisulfonate
Optical Brightener 2 is disodium 4,4′-bis-(2-sulfostyryl)biphenyl (sodium salt)
Optical Brightener 3 is Optiblanc SPL10® from 3V Sigma
Perfume encapsulate is a core-shell melamine formaldehyde perfume microcapsules
Photobleach is a sulfonated zinc phthalocyanine
Polishing enzyme is Para-nitrobenzyl esterase, reported as 1000 mg active/g
Polyetheramine as described in present disclosure.
Polymer 1 is bis((C₂H₅O)(C₂H₄O)n)(CH₃)—N⁺—CₓH₂ₓ—N⁺—(CH₃)-bis((C₂H₅O)(C₂H₄O)n), wherein n=20-30, x=3 to 8 or sulphated or sulfonated variants thereof
Polymer 2 is ethoxylated (EO₁₅) tetraethylene pentamine
Polymer 3 is ethoxylated polyethylenimine
Polymer 4 is ethoxylated hexamethylene diamine
Polymer 5 is Acusol 305, provided by Rohm&Haas
Polymer 6 is a polyethylene glycol polymer grafted with vinyl acetate side chains, provided by BASF.
Protease is Purafect Prime®, 40.6 mg active/g
Protease 2 is Savinase®, 32.89 mg active/g
Protease 3 is Purafect®, 84 mg active/g
Quaternary ammonium is C₁₂₋₁₄ Dimethylhydroxyethyl ammonium chloride
S-ACMC is Reactive Blue 19 Azo-CM-Cellulose provided by Megazyme
Soil release agent is Repel-o-tex® SF2, supplied by Solvay
Structurant is Hydrogenated Castor Oil
Violet DD is a thiophene azo polymeric hueing dye provided by Milliken

The dimensions and values disclosed herein are not to be understood as being strictly limited to the exact numerical values recited. Instead, unless otherwise specified, each such dimension is intended to mean both the recited value and a functionally equivalent range surrounding that value.

For example, a dimension disclosed as “40 mm” is intended to mean “about 40 mm.” Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cleaning composition comprising a nuclease enzyme and an amine.

2. A cleaning composition according to claim 1, wherein the nuclease enzyme is a deoxyribonuclease enzyme, a ribonuclease enzyme, or a mixture thereof.

3. A cleaning composition according to claim 1, wherein the nuclease enzyme is selected from any of E.C. classes E.C. 3.1.21.x (where x=1, 2, 3, 4, 5, 6, 7, 8, 9), 3.1.22.y (where y=1, 2, 4, 5), E.C. 3.1.30.z (where z=1, 2) or E.C. 3.1.31.1, or mixtures thereof, preferably from E.C. 3.1.21, preferably E.C. 3.1.21.1.

4. A cleaning composition according to claim 1 wherein the nuclease enzyme comprises a deoxyribonuclease enzyme.

5. A cleaning composition according to claim 1 in which the enzyme comprises an enzyme having both RNase and DNase activity, preferably being from E.C. 3.1.30.2.

6. A cleaning composition according to claim 1, wherein the nuclease enzyme is a microbial enzyme, preferably a bacterial enzyme.

7. A cleaning composition according to claim 1, wherein the enzyme has an amino acid sequence having at least 85%, or at least 90 or at least 95% or even 100% identity with the amino acid sequence shown in SEQ ID NO:1, SEQ ID NO:2 or SEQ ID NO:3.

8. A cleaning composition according to claim 1, wherein the composition further comprises a β-N-acetylglucosaminidase enzyme from E.C. 3.2.1.52, preferably an enzyme having at least 70% identity to SEQ ID NO:4.

9. A cleaning composition according to claim 1, wherein the amine is selected from the group consisting of etheramines, cyclic amines, polyamines, oligoamines, and combinations thereof.

10. A cleaning composition according to claim 9, wherein amine is a polyetheramine selected from the group consisting of polyetheramines of Formula (I), Formula (II), Formula (III), and mixtures thereof: 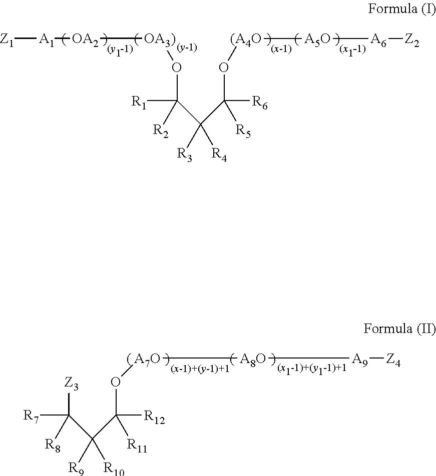
wherein each of R₁-R₁₂ is independently selected from H, alkyl, cycloalkyl, aryl, alkylaryl, or arylalkyl, wherein at least one of R₁-R₆ and at least one of R₇-R₁₂ is different from H, each of A₁-A₉ is independently selected from linear or branched alkylenes having about 2 to about 18 carbon atoms, each of Z₁-Z₄ is independently selected from OH or NH₂, wherein at least one of Z₁-Z₂ and at least one of Z₃-Z₄ is NH₂, wherein the sum of x+y is in the range of about 2 to about 200, wherein x≧1 and y≧1 and the sum of x₁+y₁ is in the range of about 2 to about 200, wherein x₁≧1 and y₁≧1; 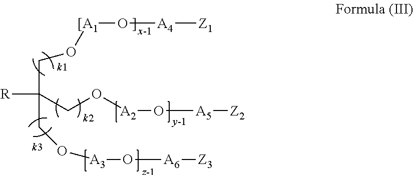
wherein
R is selected from H or a C1-C6 alkyl group, each of k₁, k₂, and k₃ is independently selected from 0, 1, 2, 3, 4, 5, or 6, each of A₁, A₂, A₃, A₄, A₅, and A₆ is independently selected from a linear or branched alkylene group having from about 2 to about 18 carbon atoms or mixtures thereof, x≧1, y≧1, and z≧1, and the sum of x+y+z is in the range of from about 3 to about 100, each of Z₁, Z₂, and Z₃ is independently selected from NH₂ or OH, where at least two of Z₁, Z₂, and Z₃ are NH₂;
and the polyetheramine has a weight average molecular weight of from about 150 to about 1000 grams/mole.

11. A composition according to claim 10, wherein in said polyetheramine of Formula (I) or Formula (II), each of A₁-A₉ is independently selected from ethylene, propylene, or butylene, preferably each of A₁-A₉ is propylene.

12. A composition according to claim 10, wherein in said polyetheramine of Formula (I) or Formula (II), each of R₁, R₂, R₅, R₆, R₇, R₈, R₁₁, and R₁₂ is H and each of R₃, R₄, R₉, and R₁₀ is independently selected from a methyl group, an ethyl group, a propyl group, a butyl group, or a phenyl group.

13. A composition according to claim 10, wherein in said polyetheramine of Formula (I) or Formula (II), each of R₃ and R₉ is an ethyl group, each of R₄ and R₁₀ is a butyl group, and each of R₁, R₂, R₅, R₆, R₇, R₈, R₁₁, and R₁₂ is H.

14. A composition according to claim 10, wherein the polyetheramine of Formula (I) has a structure according to Formula C: 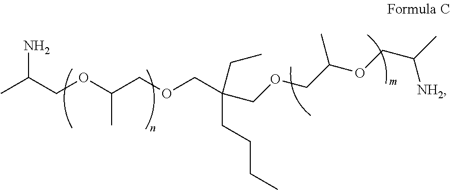
wherein n+m is from about 0 to about 8, preferably about 0 to about 6.

15. A cleaning composition according to claim 1, wherein the amine is not an alkanolamine and/or is not a polyalkyleneimine.

16. A cleaning composition according to claim 1, wherein the cleaning composition comprises from about 0.1% to about 10%, by weight of the composition, of the amine.

17. A cleaning composition according to claim 1, wherein the cleaning composition further comprises from about 1% to about 80%, by weight of the cleaning composition, of a surfactant system.

18. A cleaning composition according to claim 17, wherein the surfactant system comprises an anionic surfactant selected from the group consisting of alkyl sulfate, alkyl alkoxy sulfate, alkyl benzene sulfonate, paraffin sulfonate, and mixtures thereof.

19. A cleaning composition according to claim 1, wherein the composition further comprises fabric shading agent and/or an additional enzyme selected from lipases, amylases, proteases, mannanases, pectate lyases, cellulases, cutinases, and mixtures thereof.

20. A method of cleaning a surface, preferably a textile, comprising mixing the cleaning composition according to claim 1 with water to form an aqueous liquor and contacting a surface with the aqueous liquor in a laundering step.


SEQUENCE LISTING (6 sequences)

SEQ ID NO: 1 - protein, 109 aa, Bacillus licheniformis
ARYDDVLYFPASRYPETGAHISDAIKAGHADVCTIERSGADKRRQESLKGIPTKPGFDRDEWPMAMCEEGGKGASVRYVSSSDNRGAGSWVGNRLNGYADGTRILFIVQ

SEQ ID NO: 2 - protein, 109 aa, Bacillus subtilis
ASSYDKVLYFPLSRYPETGSHIRDAIAEGHPDICTIDDGADKRREESLKGIPTKPGYDRDEWPMAVCEEGGAGADVRYVTPSDNRGAGSWVGNQMSSYPDGTRVLFIVQ

SEQ ID NO: 3 - protein, 109 aa, Bacillus licheniformis
ARYDDILYFPASRYPETGAHISDAIKAGHSDVCTIERSGADKRRQESLKGIPTKPGFDRDEWPMAMCEEGGKGASVRYVSSSDNRGAGSWVGNRLSGFADGTRILFIVQ

SEQ ID NO: 4 - protein, 361 aa, Aggregatibacter actinomycetemcomitans
NCCVKGNSIYPQKTSTKQTGLMLDIARHFYSPEVIKSFIDTISLSGGNFLHLHFSDHENYAIESHLLNQRAENAVQGKDGIYINPYTGKPFLSYRQLDDIKAYAKAKGIELIPELDSPNHMTAIFKLVQKDRGVKYLQGLKSRQVDDEIDITNADSITFMQSLMSEVIDIFGDTSQHFHIGGDEFGYSVESNHEFITYANKLSYFLEKKGLKTRMWNDGLIKNTFEQINPNIEITYWSYDGDTQDKNEAAERRDMRVSLPELLAKGFTVLNYNSYYLYIVPKASPTFSQDAAFAAKDVIKNWDLGVWDGRNTKNRVQNTHEIAGAALSIWGEDAKALKDETIQKNTKSLLEAVIHKTNGDE

SEQ ID NO: 5 - protein, 204 aa, Aspergillus oryzae
KTGSGDSQSDPIKADLEVKGQSALPFDVDCWAILCKGAPNVLQRVNEKTKNSNRDRSGANKGPFKDPQKWGIKALPPKNPSWSAQDFKSPEEYAFASSLQGGTNAILAPVNLASQNSQGGVLNGFYSANKVAQFDPSKPQQTKGTWFQITKFTGAAGPYCKALGSNDKSVCDKNKNIAGDWGFDPAKWAYQYDEKNNKFNYVGK

SEQ ID NO: 6 - protein, 188 aa, Trichoderma harzianum
APAPMPTPPGIPTESSARTQLAGLTVAVAGSGTGYSRDLFPTWDAISGNCNAREYVLKRDGEGVQVNNACESQSGTWISPYDNASFTNASSLDIDHMVPLKNAWISGASSWTTAQREALANDVSRPQLWAVSASANRSKGDRSPDQWKPPLTSFYCTYAKSWIDVKSFYKLTITSAEKTALSSMLDTC